(19) United States
(12) Plant Patent Application Publication
(10) Pub. No.: US 2010/0071101 P1
(43) Pub. Date: Mar. 18, 2010

(54) Japanese maple plant named 'King's Blood'

(50) ;

(76) Inventor: Chris Winslow Davis, Virginia Beach, VA (US)

Correspondence Address: TROUTMAN SANDERS LLP;5200 BANK OF AMERICA PLAZA, 600 PEACHTREE STREET, N.E., SUITE 5200, ATLANTA, GA, 30308-2216, US

(21) Appl. No.: 12/283,848

(22) Filed: Sep. 15, 2008

(51) Int. Cl. A01H 5/00

(52) U.S. Cl. PLT/224

(57) ABSTRACT

A new and distinct cultivar of Japanese Maple plant named ‘King's Blood’, characterized by its brilliant scarlet foliage and leaf stems (color “Chrysanthemum Crimson 824/0” as shown in The Royal Horticultural Society Color chart, 2007 edition). King's Blood substantially retains its unique color for three seasons and does not experience warm weather foliage tip burn off is typical in other Japanese Maples.

### GENUS AND SPECIES OF PLANT CLAIMED

Acer Palmatum var. Dissectum

### VARIETAL DENOMINATION

King's Blood

### BACKGROUND OF THE INVENTION

The present invention relates to a new and distinct Cultivar of Japanese Maple Plant, botanically known as Acer Palmatum/Dissectum, and hereinafter referred to by the Cultivar name ‘King's Blood’.

### SUMMARY OF THE INVENTION

This specimen of Japanese Maple originated from a cross pollination during the spring of 2004 in Virginia Beach, Va. The parents of King's Blood are Crimson Queen (not patented) and Bloodgood (not patented). This cultivar carries favorable traits of both parents with its own unique attributes. Such include new color, resistant, and foliage. The original specimen has been carefully selected and nurtured for four years. Nine growth buds were cut from the original Japanese Maple and were reproduced by bud grafting onto Acer palmatum rootstock (a non-patented variety) in Virginia Beach, Va. in 2008.

The Japanese Maple King's Blood has a brilliant scarlet color unique to the red maple classification. Also, the Japanese Maple King's Blood has shown no warm weather serrated foliage tip burn off as may be typical in Japanese Maples, no significant greening of foliage in warmer summer climates, strong upright growth habit without brittleness, and tolerance of sunny garden locations, which in fact enhance its foliage color. The original King's Blood has been observed for four years with documentation of weather induced changes and damage characteristic with most Japanese Maples, especially of the Dissectum varieties.

### BRIEF DESCRIPTION OF THE PHOTOGRAPHS

The following color photographs illustrate the overall appearance of the Japanese Maple King's Blood. Scarlet is a great descriptive color for King's Blood foliage. (R.H.S.) colors are listed later.

FIG. 1 is color photograph of the original Japanese Maple with spring red foliage, stems, branches showing the characteristic upright branching;

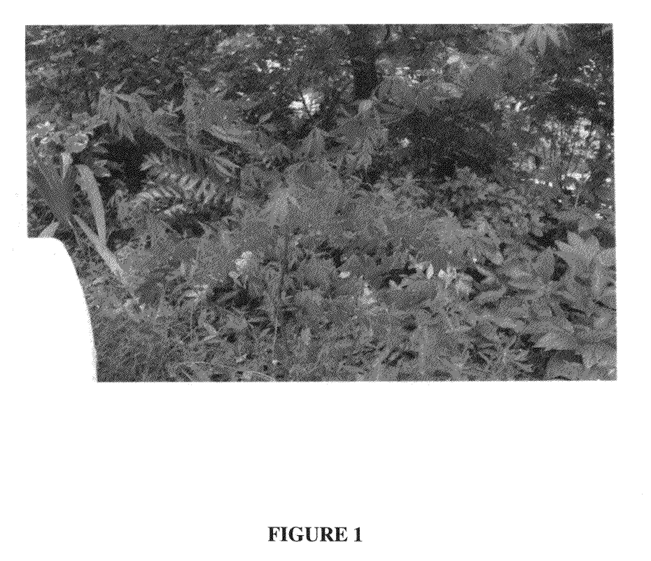

FIG. 2 is a color photograph of the Japanese Maple showing the true scarlet foliage and stems without significant green intrusion; and

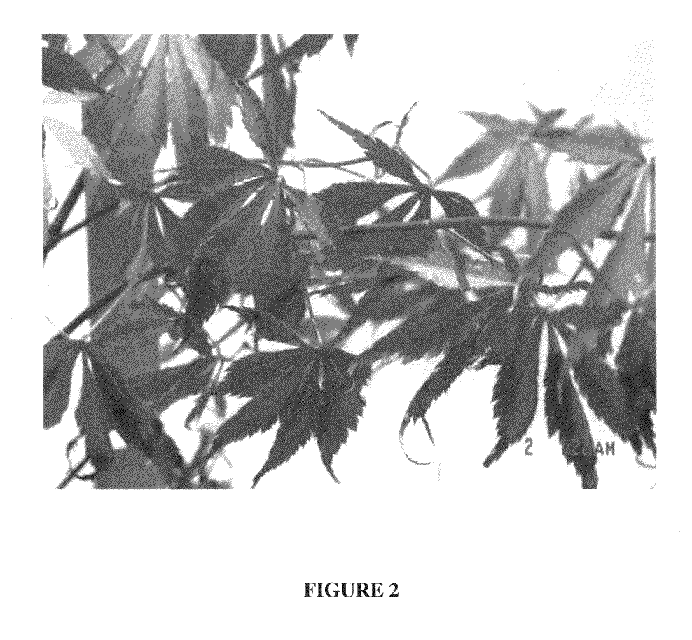

FIG. 3 is another color photograph of the original Japanese Maple showing the true scarlet foliage and stems without significant green intrusion,

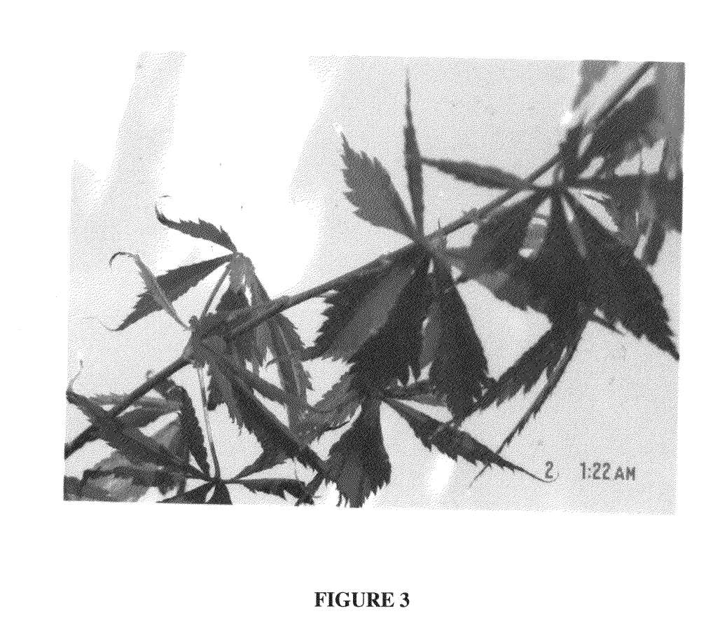

### DETAILED DESCRIPTION

This specimen of Japanese Maple originated from a cross pollination during the spring of 2004 in Virginia Beach, Va. The parents of King's Blood are Crimson Queen (not patented) and Bloodgood (not patented). This cultivar carries favorable traits of both parents and has its own unique characteristics. The original Japanese Maple was asexually reproduced by grafting cutting to Acer Palmatum Green seedling rootstock. This parent at 4 years old is approximately 42″ tall with a 30″ spread. Bark is traditional maple grayish brown without any distinguishing traits. However, branches, petioles and blades are scarlet in color.
Plant growth: Upright deciduous tree that display moderately vigorous growth habit.
Foliage: Petioles are produced opposite each other. Leave emerge with their darkest red shade “Chrysanthemum Crimson 824/0”. Color references refer to The Royal Horticultural Society Colour chart (R.H.S.) 2007 edition. The color reference “Chrysanthemum Crimson 824/0” is the closest color in the RHC, however, the actual leaves are slightly brighter in color. The foliage remains substantially this color through summer. Foliage size is approximately 3 to 4 inches long and wide, uniquely convexo-concave. As such the leaves extend beyond branch tips to form a bouquet guard of growing terminals. The leaves are dissectum lobed foliage with serrated margins and apex acute five to seven parts. Texture is normal with highly concentrated cuticle underside. Often light reflective in an orange gray haze. Foliage typically has five to seven lobes.
Summer colors: Remain the same Chrysanthemum Crimson 824/0 all year long. Except when stressed beyond normal temperature ranges above 90° at which time older leaf centers may shine orange temporarily until temperature lowers below 90° .
Fall colors: No notable change from its year round scarlet color. Same scarlet Chrysanthemum Crimson 824/0 colour prevails spring, summer and fall.
Branches and leaf stems: The branches and stems also have the same Chrysanthemum Crimson 824/0 as the foliage.
Temperature tolerances: Japanese Maples are usually grown from zones 2-8 with a natural preference towards zones 4-8. The original was grown for four seasons in Virginia Beach Va.
Flowers: The flowers of King's Blood are typical maple, inconspicuous spring blooms. The spring blooms are usually followed by twin winged seeds.
Pathogens & pest: Not confirmed as resistant to known maple wilt and/or other pathogens known to Japanese Maples. King's Blood appears to be less attractive to beetle summer infestation than its parents.
Botanical classification: Acer Palmathum Dissectum. Varietal Denomination: King's Blood.
   Parentage.—Female, cultivar Bloodgood Acer Palmatum. A maroon palmate. Male, cultivar Crimson Queen Acer Dissectum. A maroon dissected.

I hereby claim:

1. A new and distinct Japanese Maple tree named “King's Blood,” as illustrated and described.
